(19) United States
(12) Patent Application Publication
(10) Pub. No.: US 2012/0116284 A1
(43) Pub. Date: May 10, 2012

(54) Treatment of the chronic renal disease by two-stage transplantations of a patient's kidney

(76) Inventors: Vladimir Shats, Raanana (IL); Alexander Levin, Binyamina (IL)

(21) Appl. No.: 12/927,028

(22) Filed: Nov. 5, 2010

(51) Int. Cl. A61M 5/00; A61B 17/00

(52) U.S. Cl. 604/8; 128/898

(57) ABSTRACT

The invention proposes a novel method of treatment of patients with chronic kidney disease (CKD). The method includes two stage transplantations of a patient's kidney: first, one of kidneys of the patient with CKD is transplanted into a young healthy animal recipient (preferably a pig), whereas a kidney of a living person or cadaver or an animal recipient is temporarily transplanted into the body of the patient. Homeostatic factors of the animal recipient will cause the transplanted kidney recovery. In the second stage, the recovered kidney is autotransplantated back to the patient. Up to this time both the animal recipient and the patient are treated by immunosuppressive drugs.

If an allogenic kidney is transplanted into the patient's body, the patient's kidney is transplanted into the body of a young healthy pig and after it's recovery the patient's kidney re-transplanted instead of the allogenic kidney.

### STATEMENT REGARDING FEDERALLY SPONSORED RESEARCH

Not applicable.

### FIELD OF THE INVENTION

This invention relates to the field of organs' transplantation and, specifically, to kidney transplantations.

### BACKGROUND OF THE INVENTION

This invention relates to methods of treatment of chronic kidney disease (CKD), and, particularly, to methods of two-stage renal transplantations.

CKD is a serious condition associated with premature mortality, decreased quality of life, and increased health-care expenditures (Heatley, 2009). Untreated CKD can result in end-stage renal disease and necessitate dialysis or kidney transplantation. Main risk factors for CKD include acute and chronic injury, cardiovascular disease, hypertension, and especially diabetes, and obesity.

It was estimated that 16.8% of the US population >20 years old suffer from CKD (overall and by health risk factors and other characteristics). The results underscore the need to continue surveillance for CKD and its risk factors in the United States and other countries to implement new strategies to both treat and reduce the number of persons with this condition.

It is important to note that mortality rate in kidney transplantation recipients is significantly lower as compared to that in patients receiving dialysis (Barry J M, http://www.kidneyatlas.org/book5/adk5-14.ccc.OXD.pdf).

In 2003, nearly 537,000 people were on dialysis or had a functioning kidney transplant. Kidney failure now costs the U.S. healthcare system more than $25 billion every year.

Due to lack of available donors in USA, 4,573 kidney patients with CKD died in 2008. Number of kidney transplantations was 16,520, while number of patients waiting for kidney transplants was 82,364.

Currently, the new methods of treatment of CKD are under the investigation, for example, use of human growth hormones, hepatocyte growth factor, bone morphogenetic protein? (BMP7), stem cells.

Authors of the article: Li T; Surendran K; Zawaideh M A; Mathew S; Hruska K A. “Bone morphogenetic protein 7: a novel treatment for chronic renal and bone disease”. Curr Opin Nephrol Hypertension: July 2004-Volume 13-Issue 4-pp. 417-422, give a review of multiple effects of bone morphogenetic protein 7 as a potential new therapeutic agent in chronic kidney disease.

Activation of fibroblasts and myofibroblasts is believed to be the key event in the progression of CKD that leads to end-stage renal failure. Emerging evidence suggests that renal myofibroblasts may derive from tubular epithelial cells by a process of epithelial to mesenchimal transition. Experiments on mice showed that transition was stimulated by cytokines and growth factors like pro-fibrogenic transforming growth-beta factor.

Both hepatocyte growth factor and bone morphogenetic protein-7 (BMP-7) inhibit tubular epithelial cell de-differentiation, mesenchymal transformation and apoptosis stimulated by various renal injuries.

BMP-7 preserves glomerular integrity and inhibits injury-mediated mesangial matrix accumulation. In renal osteodystrophy, BMP-7 affects osteoblast morphology and number, eliminates peritrabecular fibrosis, decreases bone resorption, and increases bone formation in secondary hyperparathyroidism. Besides, BMP-7 restores normal rates of the bone formation in the degenerative bone disorders. BMP-7 is broadly efficacious in renal osteodystrophy and, importantly, increases the skeletal deposition of ingested phosphorus and calcium, improving ion homeostasis in chronic kidney disease. Also, BMP-7 was shown to prevent vascular calcification in a model of chronic kidney disease associated with the restoration of osteocalcin expression to normal tissue-restricted sites.

Treatment of CKD by BMP 7, that is capable to reverse renal fibrotic process, is described in articles of Michael Zeisberg et al.:

Reversal of experimental renal fibrosis by BMP7 provides insights into novel therapeutic strategies for chronic kidney disease. Zeisberg M, Kalluri R. (Pediatr. Nephrol. 2008 September; 23(9):1395-8. Epub 2008 Apr. 30; J. Am. Soc. Nephrol. 17: 2992-2998 pp. 2992-2998, 2006, © 2006 Amer. Soc. Nephrol).

The authors note that, “the kidney possesses an enormous capacity to regenerate following acute injury. Utilization of this endogenous regenerative capacity of the kidney in chronic disease setting could lead to repair of the diseased kidney. In addition, many forms of CKD evolve to end-stage renal failure through a series of common events that are relatively independent of the underlying insult. Blocking of these common events could then inhibit, or even reverse chronic renal injury, independent of the underlying primary disease”.

### (Frank Stutz* and Michael Zeisberg, Renal Fibroblasts and Myofibroblasts in Chronic Kidney Disease

J. Am. Soc. Nephrol. 17: 2992-2998, 2006, Amer. Soc. Nephrol.).

Reversibility of both deteriorated renal functions and fibrotic changes was discussed by other authors. It was found that following experimental kidney injury, administration of BMP-7 or inhibition of connective tissue growth factor cause striking improvement of renal function and structure (Kalluri R, Zeisberg, 2003; Nguyen T Q, Goldschmeding R, 2008). Interestingly, BMP-7, an endogenous molecule which is present in the normal kidney, vastly decreases its expression during renal injury (Kalluri R, Zeisberg). BMP-7 attenuates renal fibrosis when administered even after renal fibrosis had begun (Klahr, 2003). Remarkably, Weiss A S et al. described a patient with reversal of end-stage renal disease after aortic dissection with use of renal artery stent. Despite 2.5 months of treatment with hemodialysis, endovascular repair of renal artery stenosis reversed end-stage renal disease (http://www.biomedcentral.com/1471-2369/5/7).

In brief, there is enough data evidencing the reversibility of renal fibrotic process.

Non-transplantation methods of treatment of CKD like the administration of growth factors are very expensive, time-consuming and have serious side-effects. For example, the most common side effect of the chronic administration of growth hormone (GH) is acromegaly. Although GH increases life span, in acromegaly life span is reduced considerably. Acromegaly can cause heart enlargement, hypoglycemia with coma, excessive water retention, liver and thyroid damage.

The new evidence was found recently by Higashi K et al. (2010), they noted that the combination of pioglitazone and candesartan have additive inhibitory effect on renal fibrosis in mice, they assume that the use of this combination would be effective in the treatment of CKD although results of this study need to be confirmed.

Many researchers studied the problem of the therapeutic use of stem cells (Pleniceanu O, Harari-Steinberg O, Dekel B, Kidney Stem/Progenitor Cells: Differentiate, Sort Out or Reprogram? Stem Cells. 2010 July 22).

The authors mentioned controversies regarding the existence of a true adult kidney stem cell that highlight the importance of studying cell-based therapies using pluripotent cells, progenitor cells from fetal kidney or de-differentiated/reprogrammed adult kidney cells. Although stem cell technology and tissue engineering are potential solutions to the organ storage, xenotransplantation has generated considerable interest as a potential solution (Beshorner, http://emedicine.medscape.com/article/1014080-overview).

Nowadays, the use of pluripotent stem cells is studied intensively promising new perspectives in the future.

Xenotransplantation is considered a potential answer to the current organ shortage although the great disadvantage of allo- and, especially, xenotransplantation is rejection of transplanted organs. The future of xenotransplantation depends on the introduction of novel immunosuppressive agents that target the innate immune system, the development of clinically-applicable methods to induce donor-specific tolerance, and genetic modification of the recipients (pigs) (Ekser B et al., 2009).

An additional solution is induction of immunological tolerance state by injection of allo- or xenogeneic stem cells or bone marrow cells of the donor, especially in previously irradiated the body of a recipient in mice (Petrov R B, Immunology. Moscow, Medicine, 1982). In such a model, hematopoietic chimerism induces tolerant state in the body of the recipient (Yang Y G, 2004; Diamond P, UK Patent 2435598). The tolerance state was also induced by the implantation of the pig embryonic kidney to animal recipients (Hammermann M R, 2002).

### BRIEF SUMMARY OF THE INVENTION

This invention proposes a new surgical approach to the treatment of CKD.

There is a broad range of humoral and cellular factors involved in the pathogenesis of CKD, some of which are potential targets for treatment of the disease.

In accordance with the holistic concept of medical practice, all aspects of people's health should be taken into account; the illness is not limited by an isolated organ or system but instead it is considered the problem of the whole organism. In regard to pathogenesis of CKD, homeostatic factors of both the diseased kidney and of the organism of a patient are involved in the disease (Pino C L, Humes H D, 2010)

The method to treat CKD by transplantation of the diseased kidney of a patient into the body of young healthy animal (preferably growing pig) is linked to the capability of a body of healthy young animal recipient to produce all homeostatic factors, both humoral and cellular, influence on healing of the transplanted diseased kidney.

After recovery, a kidney is excised from the body of an animal recipient and reimplanted into the body of a patient.

Among different animals donors, pig is considered to be the best choice due to many advantages: unlimited availability of the recourse, low cost of maintenance, adequate for humans size of adult organs, moderate anatomical and physiological similarity to humans, considerable knowledge of tissue typing, considerable experience with genetic engineering, favorable public opinion (Smetanka S, Cooper DKC (2005). In Sweden, 66% of patients with CKD and 60% among the public express a positive attitude toward receiving an animal kidney xenograft (Persson O M, et al, 2001).

The idea to perform xenotransplantation into the young animals stands in accordance with some data, for example the highest rate of production of growth hormone in humans is seen at age of 20, but later the rate drops by 14% for each decade (Kareev A, http://sportfarma.ru/s_growth.htm). Also, blood levels of the growth hormone and somatomedin and their pick levels in the elderly are lower than in young persons (Florini J R et al., 1985; Vermeulin 1987). Besides, it was found that at least in articular cartilage, levels of osteogenic protein-1 that control renal branching morphogenesis (Piscione T D et al., 1970) dramatically drop with aging (Chubinskaya S et al., 2002).

The protocol of two-stages CKD transplantations according to this invention includes:

The removal of one of the diseased kidneys from a patient's body with its subsequent transplantation into the body of a recipient, preferably a healthy young pig. In the preferable modifications, the simultaneously excised kidney of a healthy pig is transplanted into the body of a patient. This procedure presents the first stage of transplantations or, to be exact, xenotransplantation.

At the end of the period of optimal recovery of the diseased patient's kidney in the body of the young healthy animal recipient, the recovered kidney is excised and re-implanted back into the patient's body. This procedure presents the second stage of the method that in fact is the autotransplantation of the recovered kidney into the patient body.

The another modification is the allotransplantion of a kidney, excised from a healthy human or cadaver, into the body of a patient. In this case, the excised diseased patient's kidney is implanted into the body of a healthy animal recipient instead of one of his kidneys excised. Following recovery, a diseased kidney is autotransplanted back into the body of a patient.

It is preferably to perform a partial resection or local cryo-ablation of the second kidney in the body of an animal recipient. The purpose of this procedure is to intensify and accelerate recovery of a diseased kidney.

A rationale of the two stage transplantations is to restore the function of the patient's diseased kidney by its transplantation into the body of a young healthy animal—with the subsequent autotransplantation of the recovered kidney back into the body of a patient. Homeostatic factors that are present in the body of a young healthy recipient will control and stimulate the recovery of the transplanted diseased kidney. Published data validate the ability of the diseased kidney to recover.

The method comprises the following main steps:

1. Preliminary check-up of the donor and animal recipient for excluding contra-indications, including diseases, bacterial and viral infections, etc.

2. Surgical removal of a kidney from a healthy animal (a pig), with its replacement in a cooling container with nutrients. In this way, the xenograft will be ready at any moment for transplantation into the body of the patient at the place of the patient's excided kidney.

3. In the case of allotransplantion, a kidney from a healthy human donor or from cadaver is transplanted into the body of the patient instead of the excised patient's kidney.

4. Following the allo- or xenotransplantation of the kidney (excised from a human donor or young healthy animal) into the body of a patient, the patient's diseased kidney is excised and implanted into the body of the animal recipient (preferably—a young healthy pig).

5. If needed, the kidney is excised from one pig but the patient's kidney is transplanted into the body of another pig.

6. Immunosuppressive treatment of both the patient and the animal recipient, has to be continued until recovery of the diseased transplanted kidney and its back autotransplantation. Within this period, proper tests including repeating biopsy of the diseased kidney in the body of the animal recipient are to be performed.

7. If signs of irreversible rejection of allo- or xenograft occur, the second attempt of transplantation is not excluded.

8. Autotransplantation of the recovered kidney from the animal into the patient's body can be performed within some months that needed for optimal recovery of a diseased kidney.

9. After completing autotransplantation the immunosuppressive treatment is stopped.

10. If following the excision of a patient's kidney transplanted to an animal recipient, the patient remains with one kidney, he begins dialysis treatment with reservation that this period should be as short as possible.

11. Following the excision of a patient's kidney, the patient's intact ureter is connected to the stump of the second ureter by the use of a T-branch and a renal artery and vein of the removed kidney are fluid communicated by a connecting pipe with an orifice. At time of autotransplantation of the patient's recovered kidney, the ureter stamp is disconnected from the T-branch and is fluid communicated with recovered kidney by a connecting pipe; in doing so, the connecting pipe with the orifice is removed and the renal artery and vein are connected again with the autotransplanted kidney. The dissected ureter is disconnected from one branch of the T-branch and is connected with said recovered diseased kidney; at the same time the free branch of the T-branch is plugged by a proper plug.

12. The increased recovery rate of the diseased kidney in the body of the young animal recipient can be achieved by partial resection or local cryo-ablation of the intact kidney in his body.

13. Another way to increase recovery rate is to administer growth factors or implant stem cells into body of the animal recipient.

14. Considering fmdings of M. Zeisberg and other authors, we can conclude that there is the matter of trade-off in our approach: the earlier in the course of CKD the xenotransplantation is performed, the higher chances that the transplanted diseased kidney will be more functional and so the higher will be chances for a diseased kidney recovering; on the contrary, in the case of the kidney with advanced CKD, chances to recover will be lower; if the graft failure occurs, a patient will come earlier to permanent dialysis. According to the published data, more than 30% of patients with CKD are referred to nephrology services later than is ideal (Heatley S A, 2009). The criteria for the assessment of CKD progression are well delineated (Strippoli et al, 2006; Merck and Merck Manual. http://www.merck.com/mmpe/sec17/ch233/ch233c.html?qt)

### DETAILED DESCRIPTION OF THE INVENTION

Removal of a diseased kidney from the body of a patient suffering from CKD is performed by routine surgical methods. At the same time a healthy kidney is excised from a healthy young animal (preferably a pig) or from a human donor, living or cadaver. The kidney of the patient is transplanted into the body of the healthy young animal and on the contrary, allo- or xenograft taken from the human donor or from the young healthy animal (a pig) is transplanted into the body of the patient. Kidneys are transplanted in the place of the excised kidneys in both patient and animal.

If the option of a kidney of a donor, either a healthy human being or an animal (a pig), is not considered to be transplanted into the body of a patient that is scheduled for dialysis treatment, the patient's renal artery and vein should be fluid communicated by a special shunt in the form of a connecting pipe with a narrow orifice designed to imitate reasonable hydraulic resistance for blood flow that passes from a renal artery to a renal vein. Aforementioned connecting pipe is removed and both renal artery and vein are again connected with stumps at time of the autotransplantation of the recovered kidney.

At time of autotransplantation of the patient's recovered kidney, the ureter stamp is disconnected from the T-branch and is fluid communicated with recovered kidney by a connecting pipe. The dissected ureter is disconnected from one branch of the T-branch and is connected with said recovered diseased kidney; at the same time the free branch of the T-branch is plugged by a proper plug.

Such an approach seems to be rational because reconstruction of the urinary tract constitutes a major source of technical complications following renal transplantation. Moreover, the immunosuppressive treatment necessitates precise technique to avoid significant morbidity and mortality in the postransplant period. Application of the connecting pipe allows prevent complications.

The disadvantage of the treatment by dialysis relates to the loss of kidney function; according to published data, recovery of renal function in patients with CKD treated by dialysis occurs in 1% of the dialysis population (Fehrman-Ekholm I, et al., 2010). So, shortening of the dialysis period—until recovery of the transplanted diseased kidney with the consequent autotransplantation—is preferable.

As was mentioned above, one kidney of an animal recipient is excised and the second one remains intact whereas in one of modifications the intact kidney may be underwent partial resection or local cryo-ablation in order to stimulate the regenerative process in the implanted patient's kidney.

Both a patient and animal recipient underwent immunosuppressive therapy until the optimal recovery of a diseased kidney in the body of the animal recipient with the subsequent excision and autotransplantion of the recovered kidney into the body of a patient.

Physiological processes of compensatory hypertrophy of a kidney, when the other kidney is removed, take usually few months. At least, the same time or more is needed for recovery of the patient's diseased kidney in the body of an animal recipient.

In order to track the process of the kidney recovery, proper tests like serial routine blood and urine tests and repeating biopsy of the diseased kidney in the body of an animal recipient are carried out.

1. A method of treatment of chronic kidney disease (CKD) consisting of the several stages:
surgical removal of a diseased kidney from a patient's body;
surgical removal of a healthy kidney from a young animal recipient with the replacement of said healthy kidney by said diseased kidney of said patient;
allo- or xenotransplatation of a kidney from the healthy human person or cadaver or said healthy animal into the body of said patient;
immunosuppressive treatment of both said patient and animal during the entire period of said diseased kidney recovery in said animal, from the allo- or xenotransplantation up to autotransplantation of said recovered kidney into the body of said patient;
proper laboratory tests are performed during all the period of recovery of said diseased kidney;

2. A method of treatment of CKD as claimed in claim 1, wherein the preferable animal is a young healthy pig.

3. A method of treatment of CKD as claimed in claim 1, wherein the diseased kidney of the patient is temporary transplanted into the animal after one of his kidneys is removed; the second recipient's (intact) kidney is underwent partial resection.

4. A method of treatment of chronic kidney disease (CKD) as claimed in claim 1, wherein the diseased kidney of the patient is temporary transplanted into the animal with removing one of its kidney; the second recipient's kidney is underwent local cryo-ablation.

5. A method of treatment of CKD as claimed in claim 1, wherein the diseased kidney of the patient is transplanted into the body of the young animal recipient; at the same time a kidney of a live human donor or cadaver is transplanted into the body of said patient.

6. A method of treatment of CKD as claimed in claim 1, wherein after completing the process of the kidney recovery in the body of the animal recipient, said recovered kidney is excised from the body of said animal and transplanted back with preceding excision of renal allo- or xenograft from the body of said patient.

7. A method of treatment of CKD as claimed in claim 5, wherein following the period of optimal recovery of the diseased kidney in the body of the animal, the allograft is excised from the body of the patient and said kidney that has been recovered in the body of said animal, is transplanted back into the body of said patient.

8. A method of treatment of chronic kidney disease (CKD) as claimed in claim 1, wherein during period of recovery of the diseased kidney in the body of the animal, the patient can be treated by hemodialysis.

9. A method of treatment of chronic kidney disease (CKD) as claimed in claim 5, wherein during period of the recovery of the kidney in the body of the animal recipient, both the patient and said animal recipient are treated by immunosuppressive therapy.

10. A method of treatment of CKD as claimed in claim 1, wherein the renal artery and vein of the temporary removed kidney of the patient, are in fluid communication by a shunt in the form of a connecting pipe with a narrow orifice designed to imitate reasonable hydraulic resistance for blood flow from said renal artery to said renal vein.

11. A method of treatment of chronic kidney disease (CKD) as claimed in claim 9, wherein on the side of the patient's intact kidney, the ureter is dissected and its both upper and lower ends are connected by a T-branch whereas the third branch of the said T-branch is connected with the stump of the ureter on the side of the removed kidney.

12. A method of treatment of chronic kidney disease (CKD) as claimed in claim 10, wherein in the process of re-transplantation of the recovered diseased kidney into the body of the patient, the dissected ureter is disconnected from one branch of the T-branch and is connected with said recovered diseased kidney; at the same time said branch of said T-branch is plugged by a proper plug.
